(19) United States
(12) Patent Application Publication
(10) Pub. No.: US 2023/0313196 A1
(43) Pub. Date: Oct. 5, 2023

(54) COMPOSITIONS AND METHODS FOR TREATING PAIN AND ANXIETY DISORDERS

(71) Applicant: John Mansell, Gillette, WY (US)

(72) Inventor: John Mansell, Gillette, WY (US)

(21) Appl. No.: 18/017,351

(22) PCT Filed: Jul. 23, 2021
(86) PCT No.: PCT/US21/43034

§ 371 Date: Jan. 20, 2023

Related U.S. Application Data
(60) Provisional application No. 63/055,496, filed on Jul. 23, 2020.

(51) Int. Cl. C12N 15/113; C12N 9/80

(52) U.S. Cl. CPC C12N 15/1137; C12N 9/80; C12Y 305/01099; C12N 2310/11

(57) ABSTRACT

Pain is often in one or more anatomical sites is the predominant complaint of subjects and is often severe enough to require medical or therapeutic intervention. In an aspect, the treatments disclosed herein affect the amount of AEA by altering FAAH expression in a bodily fluid of a subject having a pain and/or anxiety disorder. Treatment methodologies of this disclosure are characterized by a temporal reduction in the expression of the FAAH gene and secondarily a decreased expression of the FAAH enzyme or both as mediated by gene expression modifiers (GEMs).

### CROSS-REFERENCE TO RELATED APPLICATIONS

This application claims priority to U.S. Provisional Application Ser. No. 63/055,496 filed Jul. 23, 2020, and entitled “Compositions and Methods for Treating Pain and Anxiety Disorders,” which is incorporated by reference herein in its entirety.

### REFERENCE TO SEQUENCE LISTING

The content of the ASCII text file of the sequence listing named “FAAH_07232021_X_ST25.txt” which is 4.29 KB in size and was created on Jul. 23, 2021 and electronically submitted via the USPTO's “EFS-Web” patent application and document submission system herewith is incorporated herein by reference in its entirety.

### TECHNICAL FIELD

The present disclosure generally relates to compositions and methodologies for the treatment of pain and/or anxiety disorders. More specifically this disclosure relates to prophylactic and/or therapeutic utilization of oligonucleotides.

### BACKGROUND

Chronic pain and anxiety is a partially understood syndrome affecting millions of people. Generally, this syndrome is clinically diagnosed but frequently no effective treatment or cure is available. Curiously, in people who report having never suffered physical pain, higher levels of the neurotransmitter anandamide are observed which seem to express lower amounts of fatty acid amide hydrolase (FAAH), the enzyme which degrades anandamide. The structure of anandamide 100 is shown in FIG. 1. Anandamide, also known as N-arachidonoylethanolamine (AEA) is derived from arachidonic acid. It has been shown that patients who genetically are missing the ability to produce FAAH suffer little or no pain and exhibit relatively very low levels of anxiety compared to the general population or typical person.

There exists an ongoing need for compositions and methods that address the symptoms associated with chronic pain and anxiety disorders.

### BRIEF DESCRIPTION OF DRAWINGS

FIG. 1 illustrates the structure of anandamide (AEA).

FIG. 2 illustrates the structure of fatty acid amide hydrolase (FAAH).

### DETAILED DESCRIPTION

Disclosed herein are methods of treating one or more pain and anxiety disorders. Pain and anxiety disorders are defined in DSM-W and ICD-10 classifications as syndromes in which the focus of the clinical presentation is pain, anxiety or both that cause significant impairment in occupational or social function, marked distress, or both. Pain in often in one or more anatomical sites is the predominant complaint of subjects and is often severe enough to require medical or therapeutic intervention. In an aspect, the treatments disclosed herein affect the amount of AEA by altering FAAH expression in a bodily fluid of a subject having a pain and/or anxiety disorder. Treatment methodologies of this disclosure are characterized by a temporal reduction in the expression of the FAAH gene and secondarily a decreased expression of the FAAH enzyme or both as mediated by gene expression modifiers (GEMs).

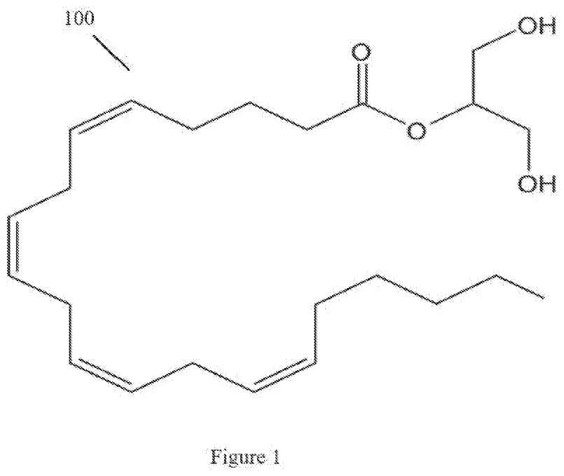

As shown in FIG. 1, anandamide (AEA) 100 is a fatty acid neurotransmitter.

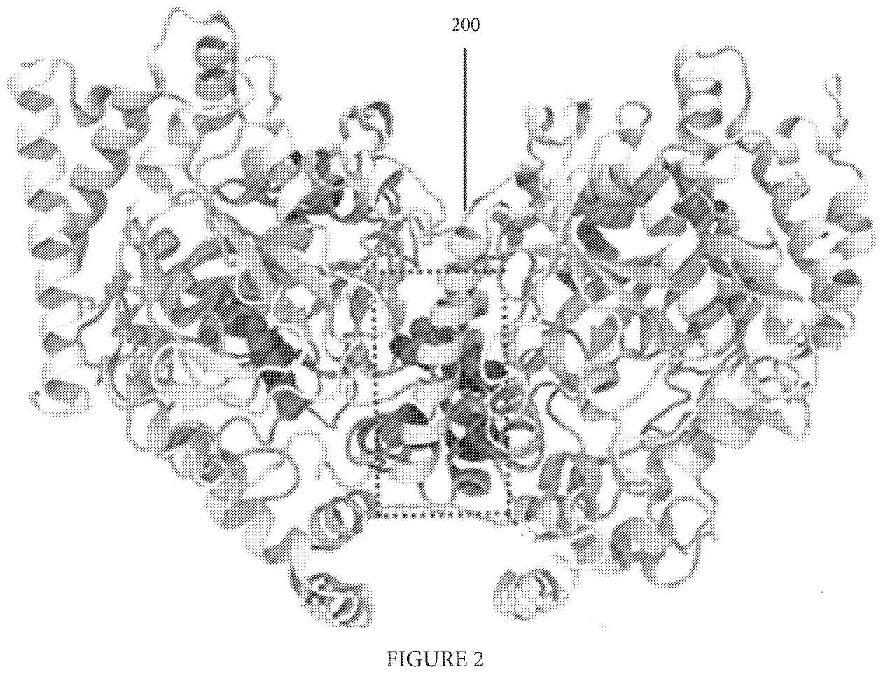

As shown in FIG. 2, fatty acid amide hydrolase (FAAH) 200 is an enzyme which may degrade anandamide (AEA) 100.

The terms “treat,” “treating,” or “treatment,” as used herein, include alleviating, abating, or ameliorating a disease or condition, or symptoms thereof; managing a disease or condition, or symptoms thereof; preventing additional symptoms; ameliorating or preventing the underlying metabolic causes of symptoms; inhibiting the disease or condition, e.g., arresting the development of the disease or condition; relieving the disease or condition; causing regression of the disease or condition; relieving a symptom caused by the disease or condition; stopping the symptoms of the disease or condition; and combination of these. Treatment as used herein also encompasses any pharmaceutical or medicinal use of the compositions herein.

The term “subject” as used herein, refers to an animal which is the object of treatment, observation, or experiment. By way of example only, a subject may be, but is not limited to, a mammal including, but not limited to, a human. In an aspect, the subject is administered the compositions disclosed herein in a therapeutically effective amount sufficient for treating, preventing, and/or ameliorating one or more symptoms of a pain or anxiety disorder. As used herein, amelioration of a symptom of the pain or anxiety disorder by administration of a particular composition of the type disclosed herein refers to any lessening of that symptom, whether lasting or transient, which can be attributed to or associated with administration of compositions of the type disclosed herein. It is contemplated that the therapeutically effective amount may be optimized by one or more healthcare professionals in consideration of the particular factors affecting a subject.

As used herein, the term “RNA interference” or “RNAi” refers to the silencing or decreasing of gene expression by iRNA agents (e.g., siRNAs, miRNAs, shRNAs), via the process of sequence-specific, post-transcriptional gene silencing in animals and plants, initiated by an iRNA agent that has a seed region sequence in the iRNA guide strand that is complementary to a sequence of the silenced gene. As used herein, the term an “iRNA agent” (abbreviation for “interfering RNA agent”), refers to an RNA agent, or chemically modified RNA, which can down-regulate the expression of a target gene. The phrase “chemical modification” as used herein refers to that meaning as is generally accepted in the art. When used with reference to the nucleic acid molecules of the present disclosure, “chemical modification” refers to any modification of the chemical structure of the nucleotides such that the resultant chemical structure differs from that of the nucleotides of native siRNA or RNA in general. For example, the term “chemical modification” encompasses the addition, substitution, or modification of native siRNA or RNA at the sugar, base, or internucleotide linkage, as described herein or as is otherwise known in the art. In certain aspects, the term “chemical modification” can refer to certain forms of RNA that are naturally occurring in certain biological systems, for example 2′-0-methyl modifications or inosine modifications. While not wishing to be bound by theory, an iRNA agent may act by one or more of a number of mechanisms, including post-transcriptional cleavage of a target mRNA, or pre-transcriptional or pre-translational mechanisms. An iRNA agent can be single-stranded (ss) or can include more than one strand, e.g., it can be a double-stranded (ds) iRNA agent. As used herein, the term “siRNA” refers to a small interfering RNA. siRNAs include short interfering RNA of about 15-60, 15-50, or 15-40 (duplex) nucleotides in length, more typically about 15-30, 15-25 or 19-25 (duplex) nucleotides in length (e.g., each complementary sequence of the double stranded siRNA is 15-60, 15-50, 15-40, 15-30, 15-25 or 19-25 nucleotides in length, and the double stranded siRNA is about 15-60, 15-50, 15-40, 15-30, 15-25 or 19-25 base pairs in length). siRNA duplexes may comprise 3′ overhangs of about 1 to about 4 nucleotides, alternatively about 2 to 3 nucleotides and 5′ phosphate termini. In some aspects, the siRNA lacks a terminal phosphate. In some aspects, one or both ends of siRNAs can include single-stranded 3′ overhangs that are two or three nucleotides in length, such as, for example, deoxythymidine (dTdT) or uracil (UU) that are not complementary to the target sequence. In some aspects, siRNA molecules can include nucleotide analogs (e.g., thiophosphate or G-clamp nucleotide analogs), alternative base linkages (e.g., phosphorothioate, phosphonoacetate, or thiophosphonoacetate) and other modifications useful for enhanced nuclease resistance, enhanced duplex stability, enhanced cellular uptake, or cell targeting.

As used herein, the GEMs need not be limited to those molecules containing only RNA but may further encompass chemically-modified nucleotides and non-nucleotides. In certain aspects, the GEMs of the present disclosure comprise separate sense and antisense sequences or regions, wherein the sense and antisense regions are covalently linked by nucleotide or non-nucleotide linking molecules as is known in the art, or are alternately non-covalently linked by ionic interactions, hydrogen bonding, Van der waals interactions, hydrophobic interactions, and/or stacking interactions. In certain aspects, the GEMs of the present disclosure comprise nucleotide sequence that is complementary to a nucleotide sequence of a target gene. In another aspect, the GEMs of the present disclosure interact with nucleotide sequence of a target gene in a manner that causes inhibition of expression of the target gene.

As used herein, “percent modification” refers to the number of nucleotides in the GEM (e.g., iRNA, or each of the strand of the siRNA or to the collective dsRNA) that have been modified. For example, a 19% modification of the antisense strand of a GEM refers to the modification of up to 4 nucleotides/bp in a 21-nucleotide sequence (21mer). A 100% modification refers to a fully modified dsRNA. The extent of chemical modification will depend upon various factors such as for example, target mRNA, off-target silencing, degree of endonuclease degradation, etc.

As used herein, the term “shRNA” or “short hairpin RNAs” refers to individual transcripts that adopt stem-loop structures which are processed into siRNA by RNAi machinery. Typical shRNA molecules comprise two inverted repeats containing the sense and antisense target sequence separated by a loop sequence. The base-paired segment may vary from 17 to 29 nucleotides, wherein one strand of the base-paired stem is complementary to the mRNA of a target gene. The loop of the shRNA stem-loop structure may be any suitable length that allows inactivation of the target gene in vivo. While the loop may be from 3 to 30 nucleotides in length, typically it is 1-10 nucleotides in length. The base paired stem may be perfectly base paired or may have 1 or 2 mismatched base pairs. The duplex portion may, but typically does not, contain one or more bulges consisting of one or more unpaired nucleotides. The shRNA may have non-base-paired 5′ and 3′ sequences extending from the base-paired stem. Typically, however, there is no 5′ extension. The first nucleotide of the shRNA at the 5′ end is a G, because this is the first nucleotide transcribed by polymerase III. If G is not present as the first base in the target sequence, a G may be added before the specific target sequence. The 5′ G typically forms a portion of the base-paired stem. Typically, the 3′ end of the shRNA is a poly U segment that is a transcription termination signal and does not form a base-paired structure. As described in the application and known to one skilled in the art, shRNAs are processed into siRNAs by the conserved cellular RNAi machinery. Thus, shRNAs are precursors of siRNAs and are, in general, similarly capable of inhibiting expression of a target mRNA transcript.

As used herein, the term “isolated” in the context of an isolated nucleic acid molecule (e.g., GEM), is one which is altered or removed from the natural state through human intervention. For example, an RNA naturally present in a living animal is not “isolated.” A synthetic RNA or dsRNA or microRNA molecule partially or completely separated from the coexisting materials of its natural state, is “isolated.”

As used herein, the term “complementary” refers to nucleic acid sequences that are capable of base-pairing according to the standard Watson-Crick complementary rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA.

As used herein, the term “gene” refers to a nucleic acid (e.g., DNA or RNA) sequence that comprises coding sequences necessary for the production of an RNA and/or a polypeptide, or its precursor as well as noncoding sequences (untranslated regions) surrounding the 5′ and 3′ ends of the coding sequences. The term “gene” encompasses both cDNA and genomic forms of a gene. A functional polypeptide can be encoded by a full-length coding sequence or by any portion of the coding sequence as long as the desired activity or functional properties (e.g., enzymatic activity, ligand binding, signal transduction, antigenic presentation) of the polypeptide are retained. The sequences which are located 5′ of the coding region and which are present on the mRNA are referred to as 5′ untranslated sequences (“5′UTR”). The sequences which are located 3′ or downstream of the coding region and which are present on the mRNA are referred to as 3′ untranslated sequences, or (“3′UTR”).

In an aspect, the compositions disclosed herein comprise a GEM which results in a down-regulation or reduction in the expression of the FAAH gene, which encodes for FAAH. In an aspect, the GEM comprises an oligonucleotide that inhibits expression of the gene coding for FAAH or alternatively substantially silences the expression of the gene coding for FAAH.

As used herein the term “substantial silencing” means that the mRNA of the targeted gene (e.g., FAAH) is inhibited and/or degraded by the presence of the introduced GEM, such that expression of the targeted gene is reduced by about 10% to 100% as compared to the level of expression seen when the GEM is not present. Generally, when a gene is substantially silenced, expression of the gene, as compared to when the GEM is not present, will be reduced by at least 40%, at least 50%, at least 60%, at least 70% (e.g., 71%, 72%, 73%, 74%, 75%, 76%, 77%, 78%, to 79%), at least 80% (e.g., 81%-84%), at least 85% (e.g., 86%, 87%, 88%, 89%), at least 90% (91%, 92%, 93%, 94%, 95%, 96%, 97%, 98%, 99%), or even 100%.

As used herein the term “substantially normal activity” means the level of expression of a gene when a GEM has not been introduced. As used herein the terms “inhibit,” “down-regulate,” or “reduce” refer to those meanings as are generally accepted in the art. With reference to exemplary nucleic acid molecules of the present disclosure, the terms generally refer the reduction in the expression of the gene, or level of RNA molecules or equivalent RNA molecules encoding one or more proteins or protein subunits, or activity of one or more proteins or protein subunits, below that observed in the absence of the nucleic acid molecules (e.g., GEM) of the present disclosure. Down-regulation can also be associated with post-transcriptional silencing, such as, RNAi-mediated cleavage, by alteration in DNA methylation patterns, or by DNA chromatin structure. Inhibition, down-regulation or reduction with a GEM can be in reference to an inactive molecule, an attenuated molecule, an oligonucleotide with a scrambled sequence, or an oligonucleotide with mismatches or alternatively, it can be in reference to the system in the absence of the oligonucleotide.

The extent of downregulation of FAAH or its respective gene product may be determined using any suitable assay. Suitable assays include without limitation, e.g., examination of protein or mRNA levels using any suitable technique such as dot blots, northern blots, in situ hybridization, ELISA, microarray hybridization, immunoprecipitation, enzyme function, as well as phenotypic assays known to those of skill in the art. To examine the extent of gene silencing, a test sample (e.g., a biological sample from organism of interest expressing the target gene(s) or a sample of cells in culture expressing the target gene(s)) is contacted with a GEM that silences, reduces, or inhibits expression of the target gene(s). Expression of the target gene in the test sample is compared to expression of the target gene in a control sample (e.g., a biological sample from organism of interest expressing the target gene or a sample of cells in culture expressing the target gene) that is not contacted with the GEM. Control samples (i.e., samples expressing the target gene) are assigned a value of 100%. In an aspect, substantial silencing, inhibition, down-regulation, or reduction of expression of a target gene is achieved when the value of the test sample relative to the control sample is about 95%, 90%, 85%, 80%, 75%, 70%, 65%, 60%, 55%, 50%, 45%, 40%, 35%, 30%, 25%, 20%, or 10%.

In an aspect the GEM is a microRNA (miRNA or miR) miRNA refers to single-stranded RNA molecules which regulate gene expression. miRNA is processed from primary transcripts known as pri-miRNA to short stem-loop structures called precursor (pre)-miRNA and finally to functional, mature miRNA. Mature miRNA molecules are partially complementary to one or more messenger RNA molecules, and their primary function is to down-regulate gene expression through the RNAi pathway.

In an aspect, the GEM is a small interfering RNA (siRNA). Naturally occurring RNAi, a double-stranded RNA (dsRNA) is cleaved by an RNase IH/helicase protein, Dicer, into small interfering RNA (siRNA) molecules, a dsRNA of 19-27 nucleotides (nt) with 2-nt overhangs at the 3′ ends. In an aspect, siRNAs are incorporated into a multicomponent-ribonuclease called RNA-induced silencing complex (RISC). One strand of siRNA remains associated with RISC and guides the complex toward a cognate RNA that has sequence complementary to the guider ss-siRNA in RISC. This siRNA-directed endonuclease digests the RNA, thereby inactivating it. These and other characteristics of RISC, siRNA molecules, and RNAi have been described.

In an aspect of the present disclosure, the GEM is an antisense oligonucleotide. Antisense oligonucleotides (ASOs) are synthetic nucleic acids that bind to a complementary target and suppress function of that target. Typically, ASOs are used to reduce or alter expression of RNA targets, particularly messenger RNA (mRNA) or microRNA (miRNA) species. As a general principle, ASOs can suppress gene expression via two different mechanisms of action, including: 1) by steric blocking, wherein the ASO tightly binds the target nucleic acid and inactivates that species, preventing its participation in cellular biology, or 2) by triggering degradation, wherein the ASO binds the target and leads to activation of a cellular nuclease that degrades the targeted nucleic acid species. One class of “target degrading” ASOs are referred to as “RNase H active.” A DNA-containing “RNase H active” ASO hybridizes with the target RNA to form heteroduplex nucleic acids that serve as a substrate for the enzyme RNase H. RNase H degrades the RNA portion of the heteroduplex molecule, thereby reducing expression of the target RNA. Degradation of the target RNA releases the ASO, which is not degraded, which is then free to recycle and can bind another RNA target of the same sequence.

In an aspect, a GEM comprises a microRNA, a siRNA, an ASO, an iRNA, an iRNA agent, a shRNA, a functional variant thereof; or combinations thereof. In some aspects, a functional variant of an oligonucleotide disclosed herein comprises at least 70% sequence identity with any sequence disclosed herein, alternatively at least 75%, alternatively at least 80%, alternatively at least 85%, alternatively at least 90% or alternatively at least 95%. In general, “identity” refers to an exact nucleotide-to-nucleotide correspondence of two oligonucleotides or polynucleotides sequences. Percent identity can be determined by a direct comparison of the sequence information between two molecules by aligning the sequences, counting the exact number of matches between the two aligned sequences, dividing by the length of the shorter sequence, and multiplying the result by 100. Readily available computer programs can be used to aid in the analysis, such as the Wisconsin Sequence Analysis Package, Version 8 (available from Genetics Computer Group, Madison, Wis.) for example, the BESTFIT, FASTA and GAP programs, which rely on the Smith and Waterman algorithm. These programs are readily utilized with the default parameters recommended by the manufacturer and described in the Wisconsin Sequence Analysis Package referred to above. For example, percent identity of a particular nucleotide sequence to a reference sequence can be determined using the homology algorithm of Smith and Waterman with a default scoring table and a gap penalty of six nucleotide positions.

Alternatively, homology can be determined by hybridization of polynucleotides under conditions which form stable duplexes between homologous regions, followed by digestion with single-stranded-specific nuclease(s), and size determination of the digested fragments. DNA sequences that are substantially homologous can be identified in a Southern hybridization experiment under, for example, stringent conditions, as defined for that particular system. Defining appropriate hybridization conditions is within the skill of the art.

As identified in the SEQUENCE LISTING (FAAH_07232021_X_ST25.txt) submitted herewith and incorporated herein by reference in its entirety (and also reproduced in TABLE 1, below), SEQ ID NO: 1 through SEQ ID NO: 28 (i.e., <210>1 through <210>28) are representative of the GEMs described herein. In an aspect, the GEM comprises a molecule having any one of SEQ ID NO: 1 through SEQ ID NO: 28, alternatively a functional variant thereof. In some aspects, a GEM suitable for use in the present disclosure comprises at least 70% sequence identity with any sequence disclosed herein, or at least 75%, or at least 80%, or at least 85%, or at least 90%, or at least 95%.

TABLE 1

| | |
|---|---|
| SEQ ID NO: 1 | cagcuggugc agaaguuaca cagua |
| SEQ ID NO: 2 | gcuggugcag aaguuacaca guaga |
| SEQ ID NO: 3 | accaauguuc cacaguccau guuca |
| SEQ ID NO: 4 | ccacagucca uguucagcua ugacu |
| SEQ ID NO: 5 | cgugaaccca uggaaguccu ccaaa |
| SEQ ID NO: 6 | ccuugcccuu cagagaagag gucua |
| SEQ ID NO: 7 | cccuucagag aagaggucua cacca |
| SEQ ID NO: 8 | cccuucagag aagaggucua cacca |
| SEQ ID NO: 9 | cccuucagag aagaggucua cacca |
| SEQ ID NO: 10 | cccuucagag aagaggucua cacca |
| SEQ ID NO: 11 | aagaagaguu gugucugcgg uucau |
| SEQ ID NO: 12 | gugaacaaag ggaccaacug u |
| SEQ ID NO: 13 | gcuguacaac ugccuggacu u |
| SEQ ID NO: 14 | guuguacaac ugacugaacu u |
| SEQ ID NO: 15 | gggaguggua uuaugaggug a |
| SEQ ID NO: 16 | ggagugguau uaugagguga u |
| SEQ ID NO: 17 | gucccaagac gacuuacaa |
| SEQ ID NO: 18 | gcagaaagga gaucauuuag u |
| SEQ ID NO: 19 | gcaucccaag aucauucuac a |
| SEQ ID NO: 20 | gccaacauug cugccaucac ugaau |
| SEQ ID NO: 21 | gagccuuucu uugacucucu gguaa |
| SEQ ID NO: 22 | agccuuucuu ugacucucug guaaa |
| SEQ ID NO: 23 | cgacuguuac aaguuugcca ucuca |
| SEQ ID NO: 24 | cacagucauc cacgggcacu guuau |
| SEQ ID NO: 25 | cgggcacugu uaugggagcu guuau |
| SEQ ID NO: 26 | acguugucuu ugaucgggcc cgaaa |
| SEQ ID NO: 27 | cgaauuggcu uugcugucag cgcuu |
| SEQ ID NO: 28 | ccacagacag augagucaac ccuca |

In an aspect, the GEM has from about 20% to about a 90% modification or alternatively from about a 40% to about 60% modification.

In an aspect, GEMs of the present disclosure (modified or unmodified) are chemically synthesized. Oligonucleotides (e.g., certain modified oligonucleotides or portions of oligonucleotides lacking ribonucleotides) are synthesized using protocols known in the art, for example as described in Caruthers et al., 1992, Methods in Enzymology 211, 3-19, Thompson et al., International PCT Publication No. WO 99/54459, Wincott et al., 1995, Nucleic Acids Res. 23, 2677-2684, Wincott et al., 1997, Methods Mol. Bio., 74, 59, Brennan et al., 1998, Biotechnol. Bioeng. 61, 33-45, and Brennan, U.S. Pat. No. 6,001,311. The synthesis of oligonucleotides makes use of common nucleic acid protecting and coupling groups, such as dimethoxytrityl at the 5′-end, and phosphoramidites at the 3′-end.

Alternatively, a GEM of the present disclosure that interacts with and down-regulates FAAH can be expressed and delivered from a transcript inserted into DNA or RNA vectors. The recombinant vectors can be DNA plasmids or viral vectors. Nonlimiting examples of GEM expressing viral vectors can be constructed based on adeno-associated virus, retrovirus, adenovirus, or alphavirus.

In some aspects, pol Hi-based constructs are used to express GEMs of the present disclosure. Transcription of the siRNA molecule sequences can be driven from a promoter for eukaryotic RNA polymerase I (pol I), RNA polymerase H (pol II), or RNA polymerase HI (pol ni), (see for example, Thompson, U.S. Pat. Nos. 5,902,880 and 6,146,886). Transcripts from pol H or pol HI promoters are expressed at high levels in all cells; the levels of a given pol II promoter in a given cell type depends on the nature of the gene regulatory sequences (enhancers, silencers, etc.) present nearby. Prokaryotic RNA polymerase promoters may also be used, providing that the prokaryotic RNA polymerase enzyme is expressed in the appropriate cells. These exemplary transcription units can be incorporated into a variety of vectors for introduction into mammalian cells, including but not restricted to, plasmid DNA vectors, viral DNA vectors (such as adenovirus or adeno-associated virus vectors), or viral RNA vectors (such as retroviral or alphavirus vectors).

Vectors used to express the GEMs of the present disclosure can encode one or both strands of a siRNA duplex, or a single self-complementary strand that self hybridizes into a siRNA duplex. The nucleic acid sequences encoding the GEMs of the present disclosure can be operably linked in a manner that allows expression of the GEM. In some aspects, the constructs comprising GEMs may additionally comprise reporter genes (e.g., green fluorescent protein) and selection genes (e.g., for antibiotic resistance). In some aspects of the present disclosure, cells (e.g., stem cells) are transfected with a construct comprising a GEM of the present disclosure. In such aspects, the construct may be configured to allow for inducible or constitutive expression of the GEM. Such cells may be introduced to a subject in order to treat a pain or anxiety-related disorder.

In an alternative aspect, the GEMs of the present disclosure are added directly to, or can be complexed with, cationic lipids, packaged within liposomes, or as a recombinant plasmid or viral vectors which express the GEM, or otherwise delivered to target cells or tissues. Nucleic acid molecules can be administered to cells by any suitable methodology, including, but not restricted to, encapsulation in liposomes, by iontophoresis, or by incorporation into other vehicles, such as biodegradable polymers, hydrogels, cyclodextrins, poly(lactic-co-glycolic)acid (PLGA) and PLCA microspheres, biodegradable nanocapsules, and bioadhesive microspheres, or by proteinaceous vectors. In one aspect, the present disclosure provides carrier systems containing the GEMs described herein. In some aspects, the carrier system is a lipid-based carrier system, cationic lipid, or liposome nucleic acid complexes, a liposome, a micelle, a virosome, a lipid nanoparticle or a mixture thereof. In other aspects, the carrier system is a polymer-based carrier system such as a cationic polymer-nucleic acid complex. In additional aspects, the carrier system is a cyclodextrin-based carrier system such as a cyclodextrin polymer-nucleic acid complex. In further aspects, the carrier system is a protein-based carrier system such as a cationic peptide-nucleic acid complex.

In other aspects, the GEM is a component of a conjugate or complex provided that can impart therapeutic activity by transferring therapeutic compounds across cellular membranes, altering the pharmacokinetics, and/or modulating the localization of the nucleic acid molecules of the present disclosure. For example, the conjugate can comprise polyethylene glycol (PEG) covalently attached to a GEM. The attached PEG can be any molecular weight, for example from about 100 to about 50,000 dal tons (Da).

In yet other aspects, the GEM is a component of compositions or formulations comprising surface-modified liposomes containing poly (ethylene glycol) lipids (e.g., PEG-modified, or long-circulating liposomes, or stealth liposomes) and GEMs. In some aspects, the siRNA molecules of the present disclosure can also be formulated or complexed with polyethyleneimine and derivatives thereof, such as polyethyleneimine-polyethyleneglycol-N-acetylgalactosamine (PEI-PEG-GAL) or polyethyleneimine-polyethyleneglycol-tri-N-acetylgalactosamine (PEI-PEG-triGAL) derivatives.

In an aspect, the GEMs of this disclosure are prepared into a composition or formulation for administration to a subject. The terms “composition” and “formulation” as used herein refer to their generally accepted meanings in the art. These terms generally refer to forms in which an agent is in a form suitable for administration (e.g., systemic or local administration) into a cell or to a subject, for example, a human, such as in a pharmaceutically acceptable carrier or diluent.

Suitable forms, in part, depend upon the use or the route of entry, for example oral, transdermal, inhalation, or by injection. Such forms should not prevent the agent from reaching a target cell (i.e., a cell into which it is desirable to deliver the nucleic acid). For example, compositions injected into the blood stream should be soluble. Other factors for consideration in determining appropriate forms include considerations such as toxicity and forms that would prevent the agent from having its intended effect. Non-limiting examples of formulations and/or compositions for use with the nucleic acid molecules of the instant present disclosure include: Lipid Nanoparticles (see for example Semple et al., 2010, Nat Biotechnol., February; 28(2): 172-6); P-glycoprotein inhibitors (such as Pluronic P85); biodegradable polymers, such as poly (DL-lactide-coglycolide) microspheres for sustained release delivery (Emerich, D F et al, 1990, Cell Transplant, 8, 47-58); and loaded nanoparticles, such as those made of polybutylcyanoacrylate. Other non-limiting examples of delivery strategies for the nucleic acid molecules of the present disclosure include those materials described in Boado et al., 1998, J. Pharm. Sci., 87, 1308-1315; Tyler et al., 1999, FEBS Lett., 421, 280-284; Partridge et al., 1995, PNAS USA., 92, 5592-5596; Boado, 1995, Adv. Drug Delivery Rev., 15, 73-107; Aldrian-Herrada et al., 1998, Nucleic Acids Res., 26, 4910-4916; and Tyler et al., 1999, PNAS USA., 96, 7053-7058. A “pharmaceutically acceptable composition” or “pharmaceutically acceptable formulation” can refer to a form that allows for the effective distribution of the nucleic acid molecules of the instant disclosure to the physical location most-suitable for their desired activity.

In an aspect, the formulation comprising the GEM further comprises an additional active agent. Examples of additional active agents for inclusion in the formulation include, but are not limited to, anesthetics, hypnotics, sedatives and sleep inducers, analgesics, antipsychotics, antidepressants, antiallergics, antianginals, anti-anxiety, antiarthritics, antiasthmatics, antidiabetics, antidiarrheal drugs,anticonvulsants, antigout drugs, antihistamines, antipruritics, emetics, antiemetics, antispasmodics, appetite suppressants, neuroactive substances, neurotransmitter agonists, antagonists, receptor blockers and reuptake modulators, beta-adrenergic blockers, calcium channel blockers, disulfuram and disulfuram-like drugs, muscle relaxants, analgesics, antipyretics, stimulants, anticholinesterase agents, parasympathomimetic agents, hormones, anticoagulants, antithrombotics, thrombolytics, immunoglobulins, immunosuppressants, hormone agonists/antagonists, vitamins, antimicrobial agents, antineoplastics, antacids, digestants, laxatives, cathartics, antiseptics, diuretics, disinfectants, fungicides, ectoparasiticides, antiparasitics, heavy metals, heavy metal antagonists, chelating agents, gases and vapors, alkaloids, salts, ions, autacoids, digitalis, cardiac glycosides, antiarrhythmics, antihypertensives, vasodilators, vasoconstrictors, antimuscarinics, ganglionic stimulating agents, ganglionic blocking agents, neuromuscular blocking agents, adrenergic nerve inhibitors, anti-oxidants, vitamins, cosmetics, anti-inflammatories, wound care products, antithrombogenic agents, antitumoral agents, antiangiogenic agents, anesthetics, antigenic agents, wound healing agents, plant extracts, growth factors, emollients, humectants, rejection/anti-rejection drugs, spermicides, conditioners, antibacterial agents, antifungal agents, antiviral agents, antibiotics, tranquilizers, cholesterol-reducing drugs, antitussives, histamine-blocking drugs, and monoamine oxidase inhibitor.

Specific compounds suitable for use in the CMBPC include silver sulfadiazine, Nystatin, Nystatin/triamcinolone, Bacitracin, nitrofurazone, nitrofurantoin, a polymyxin (e.g., Colistin, Surfactin, Polymyxin E, and Polymyxin B), doxycycline, antimicrobial peptides (e.g., natural and synthetic origin), NEOSPORIN® (i.e., Bacitracin, Polymyxin B, and Neomycin), POLYSPORIN® (i.e., Bacitracin and Polymyxin B). Additional antimicrobials include topical antimicrobials (i.e., antiseptics), examples of which include silver salts, iodine, benzalkonium chloride, alcohol, hydrogen peroxide, chlorhexidine, acetaminophen; Alfentanil Hydrochloride; Aminobenzoate Potassium; Aminobenzoate Sodium; Anidoxime; Anileridine; Anileridine Hydrochloride; Anilopam Hydrochloride; Anirolac; Antipyrine; Aspirin; Benoxaprofen; Benzydamine Hydrochloride; Bicifadine Hydrochloride; Brifentanil Hydrochloride; Bromadoline Maleate; Bromfenac Sodium; Buprenorphine Hydrochloride; Butacetin; Butixirate; Butorphanol; Butorphanol Tartrate; Carbamazepine; Carbaspirin Calcium; Carbiphene Hydrochloride; Carfentanil Citrate; Ciprefadol Succinate; Ciramadol; Ciramadol Hydrochloride; Clonixeril; Clonixin; Codeine; Codeine Phosphate; Codeine Sulfate; Conorphone Hydrochloride; Cyclazocine; Dexoxadrol Hydrochloride; Dexpemedolac; Dezocine; Diflunisal; Dihydrocodeine Bitartrate; Dimefadane; Dipyrone; Doxpicomine Hydrochloride; Drinidene; Enadoline Hydrochloride; Epirizole; Ergotamine Tartrate; Ethoxazene Hydrochloride; Etofenamate; Eugenol; Fenoprofen; Fenoprofen Calcium; Fentanyl Citrate; Floctafcnine; Flufenisal; Flunixin; Flunixin Meglumine; Flupirtine Maleate; Fluproquazone; Fluradoline Hydrochloride; Flurbiprofen; Hydromorphone Hydrochloride; Ibufenac; Indoprofen; Ketazocine; Ketorfanol; Ketorolac Tromethamine; Letimide Hydrochloride; Levomethadyl Acetate; Levomethadyl Acetate Hydrochloride; Levonantradol Hydrochloride; Levorphanol Tartrate; Lofemizole Hydrochloride; Lofentanil Oxalate; Lorcinadol; Lomoxicam; Magnesium Salicylate; Mefenamic Acid; Menabitan Hydrochloride; Meperidine Hydrochloride; Meptazinol Hydrochloride; Methadone Hydrochloride; Methadyl Acetate; Methopholine; Methotrimeprazine; Metkephamid Acetate; Mimbane Hydrochloride; Mirfentanil Hydrochloride; Molinazone; Morphine Sulfate; Moxazocine; Nabitan Hydrochloride; Nalbuphine Hydrochloride; Nalmexone Hydrochloride; Namoxyrate; Nantradol Hydrochloride; Naproxen; Naproxen Sodium; Naproxol; Nefopam Hydrochloride; Nexeridine Hydrochloride; Noracymethadol Hydrochloride; Ocfentanil Hydrochloride; Octazamide; Olvanil; Oxetorone Fumarate; Oxycodone; Oxycodone Hydrochloride; Oxycodone Terephthalate; Oxymoiphone Hydrochloride; Pemedolac; Pentamorphone; Pentazocine; Pentazocine Hydrochloride; Pentazocine Lactate; Phenazopyridine Hydrochloride; Phenyramidol Hydrochloride; Picenadol Hydrochloride; Pinadoline; Pirfenidone; Piroxicam Olamine; Pravadoline Maleate; Prodilidine Hydrochloride; Profadol Hydrochloride; Propiram Fumarate; Propoxyphene Hydrochloride; Propoxyphene Napsylate; Proxazole; Proxazole Citrate; Proxorphan Tartrate; Pyrroliphene Hydrochloride; Remifentanil Hydrochloride; Salcolex; Salethamide Maleate; Salicylamide; Salicylate Meglumine; Salsalate; Sodium S alicylate; Spiradoline Mesylate; Sufentanil; Sufentanil Citrate; Talmetacin; Talniflumate; Talosalate; Tazadolene Succinate; Tebufelone; Tetrydamine; Tifurac Sodium; Tilidine Hydrochloride; Tiopinac; Tonazocine Mesylate; Tramadol Hydrochloride; Trefentanil Hydrochloride; Trolamine; Veradoline Hydrochloride; Verilopam Hydrochloride; Volazocine; Xorphanol Mesylate; Xylazine Hydrochloride; Zenazocine Mesylate; Zomepirac Sodium; Zucapsaicin, Aflyzosin Hydrochloride; Alipamide; Althiazide; Amiquinsin Hydrochloride; Amlodipine Besylate; Amlodipine Maleate; Anaritide Acetate; Atiprosin Maleate; Belfosdil; Bemitradine; Bendacalol Mesylate; Bendroflumethiazide; Benzthiazide; Betaxolol Hydrochloride; Bethanidine Sulfate; Bevantolol Hydrochloride; Biclodil Hydrochloride; Bisoprolol; Bisoprolol Fumarate; Bucindolol Hydrochloride; Bupicomide; Buthiazide: Candoxatril; Candoxatrilat; Captopril; Carvedilol; Ceronapril; Chlorothiazide Sodium; Cicletanine; Cilazapril; Clonidine; Clonidine Hydrochloride; Clopamide; Cyclopenthiazide; Cyclothiazide; Darodipine; Debrisoquin Sulfate; Delapril Hydrochloride; Diapamide; Diazoxide; Dilevalol Hydrochloride; Diltiazem Malate; Ditekiren; Doxazosin Mesylate; Eeadotril; Enalapril Maleate; Enalaprilat; Enalkiren; Endralazine Mesylate; Epithiazide; Eprosartan; Eprosartan Mesylate; Fenoldopam Mesylate; Flavodilol Maleate; Flordipine; Flosequinan; Fosinopril Sodium; Fosinoprilat; Guanabenz; Guanabenz Acetate; Guanacline Sulfate; Guanadrel Sulfate; Guancydine; Guanethidine Monosulfate; Guanethidine Sulfate; Guanfacine Hydrochloride; Guanisoquin Sulfate; Guanoclor Sulfate; Guanoctine Hydrochloride; Guanoxabenz; Guanoxan Sulfate; Guanoxyfen Sulfate; Hydralazine Hydrochloride; Hydralazine Polistirex; Hydroflumethiazide; Indacrinone; Indapamide; Indolaprif Hydrochloride; Indoramin; Indoramin Hydrochloride; Indorenate Hydrochloride; Lacidipine; Leniquinsin; Levcromakalim; Lisinopril; Lofexidine Hydrochloride; Losartan Potassium; Losulazine Hydrochloride; Mebutamate; Mecamylamine Hydrochloride; Medroxalol; Medroxalol Hydrochloride; Methalthiazide; Methyclothiazide; Methyldopa; Methyldopate Hydrochloride; Metipranolol; Metolazone; Metoprolol Fumarate; Metoprolol Succinate; Metyrosinc; Minoxidil; Monatepil Malcate; Muzolimine; Nebivolol; Nitrendipine; Ofomine; Pargyline Hydrochloride; Pazoxide; Pelanserin Hydrochloride; Perindopril Erbumine; Phenoxybenzamine Hydrochloride; Pinacidil; Pivopril; Polythiazide; Prazo sin Hydrochloride; Primidolol; Prizidilol Hydrochloride; Quinapril Hydrochloride; Quinaprilat; Quinazosin Hydrochloride; Quinelorane Hydrochloride; Quinpirole Hydrochloride; Quinuclium Bromide; Ramipril; Rauwolfia Serpentina; Reserpine; Saprisartan Potassium; Saralasin Acetate; Sodium Nitroprusside; Sulfinalol Hydrochloride; Tasosartan; Teludipine Hydrochloride; Temocapril Hydrochloride; Terazosin Hydrochloride; Terlakiren; Tiamenidine; Tiamenidine Hydrochloride; Tierynafen; Tinabinol; Tiodazosin; Tipentosin Hydrochloride; Trichlormethiazide; Trimazosin Hydrochloride; Trimethaphan Camsylate; Trimoxamine Hydrochloride; Tripamide; Xipamide; Zankiren Hydrochloride; Zofenoprilat Arginine., Alclofenac; Alclometasone Dipropionate; Algestone Acetonide; Alpha Amylase; Ameinafal; Ameinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flaz lone; Fluazacort; Flufenamic Acid; Flumizole; Runisolide Acetate; Runixin; Runixin Meglumine; Fluocortin Butyl; Fluorometholone Acetate; Fluquazone; Rurbiprofen; Ruretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lomoxicam; Loteprednol Etabonate; Meclofenamate Sodium; Meclofenamic Acid; Meclorisone Dibutyrate; Mefenamic Acid; Mesalamine; Meseclazone; Methylprednisolone Suleptanate; Momiflumate; Nabumetone; Naproxen; Naproxen Sodium; Naproxol; Nimazone; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrydamine; Tiopinac; Tixocortol Pivalate; Tolmetin; Tolmetin Sodium; Triclonide; Triflumidate; Zidometacin; and Zomepirac Sodium.

In an aspect, the composition and/or formulation may contain additional ingredients. As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin or anandamide itself; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials.

In a particular aspect, a subject who has been diagnosed with a pain or anxiety disorder such as fibromyalgia or complex regional pain syndrome is treated using a formulation and/or composition comprising a GEM of the type disclosed herein. The formulation and/or composition comprising the GEM is introduced to a tender point of the subject. Herein, a “tender point” refers to an area of tenderness occurring in muscle, a muscle-tendon junction, bursa, or fat pad. In an aspect, the tender points comprise (1) low cervical region: Front neck area just below the chin near the C5-C7 vertebrae; (2) second rib: front chest area below the collarbone about 2 inches from the shoulder joint; (3) occiput: back of the neck at the base of the skull; (4) trapezius muscle: back shoulder area where this large muscle drapes over the top of the shoulder; (5) supraspinatus muscle: Shoulder blade area just at the top of the shoulder blade; (6) lateral epicondyle: elbow area in the inside of the arm crease; (7) gluteal: rear end at upper outer quadrant of the buttocks; (8) greater trochanter: rear hip in the back and (9) knee: knee area on the inside where the fat pad sits.

In such an aspect, the GEM comprises a FAAH expression inhibitor. In another aspect, the GEM is formulated with a CSF mimic.

Without wishing to be limited by theory, these different forms of oligonucleotides would diminish efficient transcription of FAAH mRNA, reduce successful movement of guide strand mRNA to translation and interfere with efficient translation of mRNA which produces FAAH. In an aspect, a GEM for the present disclosure that reduces FAAH protein and/or mRNA levels in the bodily fluid of a subject has any one of SEQ ID NO: 1 through SEQ ID NO: 28.

Conventional methods, known to those of ordinary skill in the art of medicine, can be used to administer the pharmaceutical formulations to a mammalian subject. The pharmaceutical formulations can be administered via oral, subcutaneous, intrapulmonary, transmucosal, intraperitoneal, intrauterine, sublingual, intrathecal, or intramuscular routes.

In a specific aspect, a formulation comprises a gene expression modifier selected from the group consisting of an oligonucleotide having any of SEQ ID NO: 1 through SEQ ID NO: 28 and a cerebrospinal fluid compatible diluent. The formulation may further comprise a functional variant of the gene expression modifier.

Additionally or alternatively, in a specific aspect, a method of treating a subject diagnosed with a pain or anxiety disorder comprises introducing into the subject's cerebrospinal fluid a composition comprising an oligonucleotide that reduces the expressed amount of intracellular FAAH of the concentration of FAAH in the subject's cerebrospinal fluid. The pain or anxiety disorder may be fibromyalgia, complex regional pain or only an anxiety disorder without associated pain.

Additionally or alternatively, in a specific aspect, a method of treating a subject diagnosed with a pain disorder comprises introducing to a plurality of tender points of a subject a gene expression modifier that reduces the concentration of calcitonin gene-related protein. The oligonucleotide may be selected from the group consisting of an oligonucleotide having any of SEQ ID NO: 1 through SEQ ID NO: 28. The oligonucleotide may have at least 70% sequence homology to any of SEQ ID NO: 1 through SEQ ID NO: 28. The oligonucleotide may comprise SEQ ID NO: 1. The pain disorder may be fibromyalgia. The pain disorder may be complex regional pain syndrome.

Additionally or alternatively, in a specific aspect, a method of treating a subject diagnosed with a pain or anxiety disorder comprises introducing into cells a construct comprising a reporter gene and a gene expression modifier into a bodily fluid of the subject. The pain disorder may be fibromyalgia. The pain disorder may be complex regional pain syndrome. The construct may comprise a pol II vector. The gene expression modifier may be selected from the group consisting of an oligonucleotide having any of SEQ ID No. 1 through SEQ ID NO: 28.

Additionally or alternatively, in a specific aspect, use of a composition comprising an oligonucleotide for treatment of a subject diagnosed with a pain or anxiety disorder, comprises introducing into subject's cerebrospinal fluid the composition comprising the oligonucleotide to reduce expression of FAAH and increase the concentration of AEA in the subject's cerebrospinal fluid. The oligonucleotide may be a gene expression modifier selected from the group having any of SEQ ID NO: 1 to SEQ ID NO: 28. The oligonucleotide may have at least 70% sequence homology to any of SEQ ID NO: 1 through SEQ ID NO: 28. The pain disorder may be fibromyalgia. The pain disorder may be complex regional pain syndrome. The anxiety disorder may or may not be associated with a pain disorder.

Additionally or alternatively, in a specific aspect, use of a gene expression modifier for treatment of a subject diagnosed with a pain disorder comprises introducing into a plurality of tender points of the subject a gene expression modifier to reduce the concentration of calcitonin gene-related protein. The oligonucleotide may be selected from the group consisting of an oligonucleotide having any of SEQ ID NO: 1 through SEQ ID NO: 28. The oligonucleotide may have at least 70% sequence homology to any of SEQ ID NO: 1 through SEQ ID NO: 28. The pain disorder may be fibromyalgia. The pain disorder may be complex regional pain syndrome. The anxiety disorder may or may not be associated with a pain disorder.

In a specific aspect, use of a construct comprising a reporter gene and a gene expression modifier in the treatment of a subject diagnosed with a pain or anxiety disorder, comprises introducing into cells the construct comprising the reporter gene and the gene expression modifier into a bodily fluid of the subject. The pain disorder may be fibromyalgia. The pain disorder may be complex regional pain syndrome. The anxiety disorder may or may not be associated with a pain disorder. The construct may comprise a pol II vector. The gene expression modifier may be selected from the group consisting of an oligonucleotide having any of SEQ ID NO:1 through SEQ ID NO: 28.

In an aspect, a subject is administered a GEM of the type disclosed herein (e.g., microRNAs) intrathecally via infusion into the CSF or via implantation with transduced GEM-secreting cells (e.g., secreting microRNAs of the type disclosed herein). In some aspects of the present disclosure, the GEM is administered via utilization of an intrathecal pump. A formulation of the GEM for intrathecal administration may further comprise a CSF compatible diluent such as a sterile isotonic solution that can be injected in a volume ranging from about 0.5 ml to about 5 ml. Without wishing to be limited by theory, such GEMS may enter the central nervous system cell membranes due to their small size and other fundamental characteristics. mRNA has been found abundantly in whole blood, plasma, and interstitial fluids. Once the therapeutic mRNA and oligopeptides are intra-cellular, affinity dynamics with the components of the nuclear, cytoplasmic, and ribosomal elements of the FAAH production chain will determine their effectiveness in reducing production of FAAH and decreasing the intensity of syndrome-associated symptoms.

Injectable formulations of the GEM compositions or formulations of the present disclosure may contain various carriers. Physiologically acceptable excipients may include, for example, 5% dextrose, 0.9% saline, Ringer's solution, or other suitable excipients. Intramuscular preparations, e.g., a sterile formulation of the compounds of the present disclosure can be dissolved and administered in a pharmaceutical excipient was as water-for-injection, 0.9% saline, or 5% glucose solution.

### Prophetic Example

The following prophetic example is given as a particular aspect of the present disclosure and to demonstrate the practice and advantages thereof. It is understood that the example is given by way of illustration and is not intended to limit the specification or the claims to follow in any manner.

A culture of human peripheral blood monocytes/macrophages will be established using standard techniques. Certain cell types like glial cells may be cultured in four-well plates may be assayed for specific production of FAAH such as by using an ELISA assay for either material. For example, a culture of monocytes/macrophages and/or monocyte-derived macrophages may be incubated with an appropriate trigger (e.g., capsaicin) and the level of FAAH or mRNA thereof assayed using any suitable methodology. Alternatively, supernatant solutions of AEA may be tested over time to measure the rate of degradation.

The extent to which FAAH are present can be determined by a suitable analytical assay, both in terms of total concentration and concentration as a function of time from the introduction of a trigger. The monocytes/macrophages in the absence of a GEM are expected to show an increase in the expression at both the FAAH protein and mRNA level of FAAH. In the presence of a GEM, reduction in the amount of mRNA or protein produced for FAAH is expected. In the presence of a GEM, a latency in the time for increased production of FAAH may also be observed.

The following embodiments (1-54) are exemplary of the instantly disclosed concepts.

1. A formulation comprising a gene expression modifier selected from a group consisting of an oligonucleotide having any of SEQ ID NO: 1 through SEQ ID NO: 28.

2. The formulation of embodiment 1, further comprising a cerebrospinal fluid compatible diluent.

3. The formulation of embodiment 1, wherein the oligonucleotide comprises at least 70% sequence identity to any of SEQ ID NO: 1 through SEQ ID NO: 28.

4. The formulation of embodiment 1, wherein the oligonucleotide comprises at least 75% sequence identity to any of SEQ ID NO: 1 through SEQ ID NO: 28.

5. The formulation of embodiment 1, wherein the oligonucleotide comprises at least 80% sequence identity to any of SEQ ID NO: 1 through SEQ ID NO: 28.

6. The formulation of embodiment 1, wherein the oligonucleotide comprises at least 85% sequence identity to any of SEQ ID NO: 1 through SEQ ID NO: 28.

7. The formulation of embodiment 1, wherein the oligonucleotide comprises at least 90% sequence identity to any of SEQ ID NO: 1 through SEQ ID NO: 28.

8. The formulation of embodiment 1, wherein the oligonucleotide comprises at least 95% sequence identity to any of SEQ ID NO: 1 through SEQ ID NO: 28.

9. The composition of embodiment 1, wherein the composition is formulated for delivery to a subject.

10. The formulation of embodiment 2, wherein the oligonucleotide comprises at least 70% sequence identity to any of SEQ ID NO: 1 through SEQ ID NO: 28.

11. The formulation of embodiment 2, wherein the oligonucleotide comprises at least 75% sequence identity to any of SEQ ID NO: 1 through SEQ ID NO: 28.

12. The formulation of embodiment 2, wherein the oligonucleotide comprises at least 80% sequence identity to any of SEQ ID NO: 1 through SEQ ID NO: 28.

13. The formulation of embodiment 2, wherein the oligonucleotide comprises at least 85% sequence identity to any of SEQ ID NO: 1 through SEQ ID NO: 28.

14. The formulation of embodiment 2, wherein the oligonucleotide comprises at least 90% sequence identity to any of SEQ ID NO: 1 through SEQ ID NO: 28.

15. The formulation of embodiment 2, wherein the oligonucleotide comprises at least 95% sequence identity to any of SEQ ID NO: 1 through SEQ ID NO: 28.

16. The composition of embodiment 2, wherein the composition is formulated for delivery to a subject.

17. A method for affecting an amount of anandamide comprising mediating via a gene expression modifier.

18. The method of embodiment 17, further comprising temporally reducing expression of a fatty acid amide hydrolase (“FAAH”) gene.

19. The method of embodiment 17, further comprising decreasing expression of fatty acid amide hydrolase (“FAAH”).

20. The method of embodiment 17, wherein the gene expression modifier comprises at least 70% sequence identity to any of SEQ ID NO: 1 through SEQ ID NO: 28.

21. The method of embodiment 17, wherein the gene expression modifier comprises an oligonucleotide comprising at least 70% sequence identity to any of SEQ ID NO: 1 through SEQ ID NO: 28.

22. The method of embodiment 17, wherein the gene expression modifier comprises at least 75% sequence identity to any of SEQ ID NO: 1 through SEQ ID NO: 28.

23. The method of embodiment 17, wherein the gene expression modifier comprises an oligonucleotide comprising at least 75% sequence identity to any of SEQ ID NO: 1 through SEQ ID NO: 28.

24. The method of embodiment 17, wherein the gene expression modifier comprises at least 80% sequence identity to any of SEQ ID NO: 1 through SEQ ID NO: 28.

The method of embodiment 17, wherein the gene expression modifier comprises an oligonucleotide comprising at least 80% sequence identity to any of SEQ ID NO: 1 through SEQ ID NO: 28.

25. The method of embodiment 17, wherein the gene expression modifier comprises at least 85% sequence identity to any of SEQ ID NO: 1 through SEQ ID NO: 28.

26. The method of embodiment 17, wherein the gene expression modifier comprises an oligonucleotide comprising at least 85% sequence identity to any of SEQ ID NO: 1 through SEQ ID NO: 28.

27. The method of embodiment 17, wherein the gene expression modifier comprises at least 90% sequence identity to any of SEQ ID NO: 1 through SEQ ID NO: 28.

28. The method of embodiment 17, wherein the gene expression modifier comprises an oligonucleotide comprising at least 90% sequence identity to any of SEQ ID NO: 1 through SEQ ID NO: 28.

29. The method of embodiment 17, wherein the gene expression modifier comprises at least 95% sequence identity to any of SEQ ID NO: 1 through SEQ ID NO: 28.

30. The method of embodiment 17, wherein the gene expression modifier comprises an oligonucleotide comprising at least 95% sequence identity to any of SEQ ID NO: 1 through SEQ ID NO: 28.

31. The method of embodiment 18, wherein the gene expression modifier comprises at least 70% sequence identity to any of SEQ ID NO: 1 through SEQ ID NO: 28.

32. The method of embodiment 18, wherein the gene expression modifier comprises an oligonucleotide comprising at least 70% sequence identity to any of SEQ ID NO: 1 through SEQ ID NO: 28.

33. The method of embodiment 18, wherein the gene expression modifier comprises at least 75% sequence identity to any of SEQ ID NO: 1 through SEQ ID NO: 28.

34. The method of embodiment 18, wherein the gene expression modifier comprises an oligonucleotide comprising at least 75% sequence identity to any of SEQ ID NO: 1 through SEQ ID NO: 28.

35. The method of embodiment 18, wherein the gene expression modifier comprises at least 80% sequence identity to any of SEQ ID NO: 1 through SEQ ID NO: 28.

36. The method of embodiment 18, wherein the gene expression modifier comprises an oligonucleotide comprising at least 80% sequence identity to any of SEQ ID NO: 1 through SEQ ID NO: 28.

37. The method of embodiment 18, wherein the gene expression modifier comprises at least 85% sequence identity to any of SEQ ID NO: 1 through SEQ ID NO: 28.

38. The method of embodiment 18, wherein the gene expression modifier comprises an oligonucleotide comprising at least 85% sequence identity to any of SEQ ID NO: 1 through SEQ ID NO: 28.

39. The method of embodiment 18, wherein the gene expression modifier comprises at least 90% sequence identity to any of SEQ ID NO: 1 through SEQ ID NO: 28.

40. The method of embodiment 18, wherein the gene expression modifier comprises an oligonucleotide comprising at least 90% sequence identity to any of SEQ ID NO: 1 through SEQ ID NO: 28.

41. The method of embodiment 18, wherein the gene expression modifier comprises at least 95% sequence identity to any of SEQ ID NO: 1 through SEQ ID NO: 28.

42. The method of embodiment 18, wherein the gene expression modifier comprises an oligonucleotide comprising at least 95% sequence identity to any of SEQ ID NO: 1 through SEQ ID NO: 28.

43. The method of embodiment 19, wherein the gene expression modifier comprises at least 70% sequence identity to any of SEQ ID NO: 1 through SEQ ID NO: 28.

44. The method of embodiment 19, wherein the gene expression modifier comprises an oligonucleotide comprising at least 70% sequence identity to any of SEQ ID NO: 1 through SEQ ID NO: 28.

45. The method of embodiment 19, wherein the gene expression modifier comprises at least 75% sequence identity to any of SEQ ID NO: 1 through SEQ ID NO: 28.

46. The method of embodiment 19, wherein the gene expression modifier comprises an oligonucleotide comprising at least 75% sequence identity to any of SEQ ID NO: 1 through SEQ ID NO: 28.

47. The method of embodiment 19, wherein the gene expression modifier comprises at least 80% sequence identity to any of SEQ ID NO: 1 through SEQ ID NO: 28.

48. The method of embodiment 19, wherein the gene expression modifier comprises an oligonucleotide comprising at least 80% sequence identity to any of SEQ ID NO: 1 through SEQ ID NO: 28.

49. The method of embodiment 19, wherein the gene expression modifier comprises at least 85% sequence identity to any of SEQ ID NO: 1 through SEQ ID NO: 28.

50. The method of embodiment 19, wherein the gene expression modifier comprises an oligonucleotide comprising at least 85% sequence identity to any of SEQ ID NO: 1 through SEQ ID NO: 28.

51. The method of embodiment 19, wherein the gene expression modifier comprises at least 90% sequence identity to any of SEQ ID NO: 1 through SEQ ID NO: 28.

52, The method of embodiment 19, wherein the gene expression modifier comprises an oligonucleotide comprising at least 90% sequence identity to any of SEQ ID NO: 1 through SEQ ID NO: 28.

53. The method of embodiment 19, wherein the gene expression modifier comprises at least 95% sequence identity to any of SEQ ID NO: 1 through SEQ ID NO: 28.

54. The method of embodiment 19, wherein the gene expression modifier comprises an oligonucleotide comprising at least 95% sequence identity to any of SEQ ID NO: 1 through SEQ ID NO: 28.

One of skill in the art, upon viewing this disclosure, will appreciate one or more additional aspects and/or variations of the subject matter disclosed herein. As such, the forgoing is in no way intended to be limited to the aspect or example disclosed herein. Variations, combinations, and/or modifications of the aspects disclosed herein made by a person having ordinary skill in the art are within the scope of the disclosure. Alternative aspects that result from combining, integrating, and/or omitting features of the aspects are also within the scope of the disclosure. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.). For example, whenever a numerical range with a lower limit, R1, and an upper limit, Ru, is disclosed, any number falling within the range is specifically disclosed. In particular, the following numbers within the range are specifically disclosed: R=R1+k*(Ru−Rl), wherein k is a variable ranging from 1 percent to 100 percent with a 1 percent increment, i.e., k is 1 percent, 2 percent, 3 percent, 4 percent, 5 percent, . . . , 50 percent, 51 percent, 52 percent, . . . , 95 percent, 96 percent, 97 percent, 98 percent, 99 percent, or 100 percent. Moreover, any numerical range defined by two R numbers as defined in the above is also specifically disclosed. Use of the term “optionally” with respect to any element of a claim means that the element is required, or alternatively, the element is not required, both alternatives being within the scope of the claim. Use of broader terms such as “comprises,” “includes,” and “having” should be understood to provide support for narrower terms such as “consisting of,” “consisting essentially of,” and “comprised substantially of.”

Accordingly, the scope of protection is not limited by the description set out above but is defined by the claims that follow, that scope including all equivalents of the subject matter of the claims. Each and every claim is incorporated as further disclosure into the specification and the claims are aspects of the presently disclosed subject matter.

What is claimed is:

1. A formulation comprising a gene expression modifier selected from a group consisting of an oligonucleotide having any of SEQ ID NO: 1 through SEQ ID NO: 28.

2. The formulation of claim 1, further comprising a cerebrospinal fluid compatible diluent.

3. The formulation of claim 1, wherein the oligonucleotide comprises at least 70% sequence identity to any of SEQ ID NO: 1 through SEQ ID NO: 28.

4. The formulation of claim 1, wherein the oligonucleotide comprises at least 75% sequence identity to any of SEQ ID NO: 1 through SEQ ID NO: 28.

5. The formulation of claim 1, wherein the oligonucleotide comprises at least 80% sequence identity to any of SEQ ID NO: 1 through SEQ ID NO: 28.

6. The formulation of claim 1, wherein the oligonucleotide comprises at least 85% sequence identity to any of SEQ ID NO: 1 through SEQ ID NO: 28.

7. The formulation of claim 1, wherein the oligonucleotide comprises at least 90% sequence identity to any of SEQ ID NO: 1 through SEQ ID NO: 28.

8. The formulation of claim 1, wherein the oligonucleotide comprises at least 95% sequence identity to any of SEQ ID NO: 1 through SEQ ID NO: 28.

9. The composition of claim 1, wherein the composition is formulated for delivery to a subject.

10. A method for affecting an amount of anandamide comprising mediating via a gene expression modifier.

11. The method of claim 10, further comprising temporally reducing expression of a fatty acid amide hydrolase (“FAAH”) gene.

12. The method of claim 10, further comprising decreasing expression of fatty acid amide hydrolase (“FAAH”).

13. The method of claim 10, wherein the gene expression modifier comprises at least 70% sequence identity to any of SEQ ID NO: 1 through SEQ ID NO: 28.

14. The method of claim 10, wherein the gene expression modifier comprises an oligonucleotide comprising at least 70% sequence identity to any of SEQ ID NO: 1 through SEQ ID NO: 28.

15. The method of claim 10, wherein the gene expression modifier comprises at least 75% sequence identity to any of SEQ ID NO: 1 through SEQ ID NO: 28.

16. The method of claim 10, wherein the gene expression modifier comprises at least 80% sequence identity to any of SEQ ID NO: 1 through SEQ ID NO: 28.

17. The method of claim 10, wherein the gene expression modifier comprises at least 85% sequence identity to any of SEQ ID NO: 1 through SEQ ID NO: 28.

18. The method of claim 10, wherein the gene expression modifier comprises at least 90% sequence identity to any of SEQ ID NO: 1 through SEQ ID NO: 28.

19. The method of claim 10, wherein the gene expression modifier comprises at least 95% sequence identity to any of SEQ ID NO: 1 through SEQ ID NO: 28.

20. The method of claim 10, wherein the gene expression modifier comprises an oligonucleotide comprising at least 95% sequence identity to any of SEQ ID NO: 1 through SEQ ID NO: 28.


SEQUENCE LISTING (28 sequences)

SEQ ID NO: 1 - RNA, 25 nt, Homo sapiens
cagcuggugcagaaguuacacagua

SEQ ID NO: 2 - RNA, 25 nt, Homo sapiens
gcuggugcagaaguuacacaguaga

SEQ ID NO: 3 - RNA, 25 nt, Homo sapiens
accaauguuccacaguccauguuca

SEQ ID NO: 4 - RNA, 25 nt, Homo sapiens
ccacaguccauguucagcuaugacu

SEQ ID NO: 5 - RNA, 25 nt, Homo sapiens
cgugaacccauggaaguccuccaaa

SEQ ID NO: 6 - RNA, 25 nt, Homo sapiens
ccuugcccuucagagaagaggucua

SEQ ID NO: 7 - RNA, 25 nt, Homo sapiens
cccuucagagaagaggucuacacca

SEQ ID NO: 8 - RNA, 25 nt, Homo sapiens
cccuucagagaagaggucuacacca

SEQ ID NO: 9 - RNA, 25 nt, Homo sapiens
cccuucagagaagaggucuacacca

SEQ ID NO: 10 - RNA, 25 nt, Homo sapiens
cccuucagagaagaggucuacacca

SEQ ID NO: 11 - RNA, 25 nt, Homo sapiens
aagaagaguugugucugcgguucau

SEQ ID NO: 12 - RNA, 21 nt, Homo sapiens
gugaacaaagggaccaacugu

SEQ ID NO: 13 - RNA, 21 nt, Homo sapiens
gcuguacaacugccuggacuu

SEQ ID NO: 14 - RNA, 21 nt, Homo sapiens
guuguacaacugacugaacuu

SEQ ID NO: 15 - RNA, 21 nt, Homo sapiens
gggagugguauuaugagguga

SEQ ID NO: 16 - RNA, 21 nt, Homo sapiens
ggagugguauuaugaggugau

SEQ ID NO: 17 - RNA, 19 nt, Homo sapiens
gucccaagacgacuuacaa

SEQ ID NO: 18 - RNA, 21 nt, Homo sapiens
gcagaaaggagaucauuuagu

SEQ ID NO: 19 - RNA, 21 nt, Homo sapiens
gcaucccaagaucauucuaca

SEQ ID NO: 20 - RNA, 25 nt, Homo sapiens
gccaacauugcugccaucacugaau

SEQ ID NO: 21 - RNA, 25 nt, Homo sapiens
gagccuuucuuugacucucugguaa

SEQ ID NO: 22 - RNA, 25 nt, Homo sapiens
agccuuucuuugacucucugguaaa

SEQ ID NO: 23 - RNA, 25 nt, Homo sapiens
cgacuguuacaaguuugccaucuca

SEQ ID NO: 24 - RNA, 25 nt, Homo sapiens
cacagucauccacgggcacuguuau

SEQ ID NO: 25 - RNA, 25 nt, Homo sapiens
cgggcacuguuaugggagcuguuau

SEQ ID NO: 26 - RNA, 25 nt, Homo sapiens
acguugucuuugaucgggcccgaaa

SEQ ID NO: 27 - RNA, 25 nt, Homo sapiens
cgaauuggcuuugcugucagcgcuu

SEQ ID NO: 28 - RNA, 25 nt, Homo sapiens
ccacagacagaugagucaacccuca